(19) United States
(12) Patent Application Publication
(10) Pub. No.: US 2007/0100225 A1
(43) Pub. Date: May 3, 2007

(54) Medical imaging modality

(76) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/546,506

(22) Filed: Oct. 11, 2006

(30) Foreign Application Priority Data
Oct. 12, 2005 (DE) 10 2005 048 853.6

(51) Int. Cl. A61B 5/05

(52) U.S. Cl. 600/407; 600/436

(57) ABSTRACT

A medical imaging modality with a PET detector ring for Positron Emission Tomography connected on the data side to a PET image processing unit is to be structured in such a way that, on the basis of the images generated in the modality, a reliable detection and precise localization of metabolism anomalies, especially of malign tissue with tumor incidence is possible. The modality is further intended to offer good access to the patient, so that operative or minimally-invasive interventions on the patient can be undertaken and checked alongside the image recording. To this end there is provision, in accordance with the invention, for an ACT recording device for angiographic computer tomography connected on the data side to an ACT image processing unit to be arranged adjacent to the PET detector, whereby a common display unit for display of PET images and/or ACT images generated in the relevant image processing unit is assigned to the PET image processing unit and the ACT image processing unit.

### CROSS REFERENCE TO RELATED APPLICATIONS

This application claims priority of German application No. 10 2005 048 853.6 filed Oct. 12, 2005, which is incorporated by reference herein in its entirety.

### FIELD OF THE INVENTION

The invention relates to a medical imaging modality with a PET detector ring for Positron Emission Tomography connected on the data side to a PET image processing unit.

### BACKGROUND OF THE INVENTION

Diseases of the vessels such as strokes, aneurysm and abdominal aorta aneurysms are among the most frequently occurring diseases in the world. With these diseases, but also especially with tumor diseases or similar, rapid and secure diagnosis and immediate initiation of therapy are of particular significance for the recovery process.

The diagnosis of these types of diseases is supported by imaging methods. One imaging method which can supply a high level of diagnostic information is Positron Emission Tomography (PET). The method is based on the representation of the distribution of a radioactively marked substance, of a so-called radiopharmacon or tracer, in the organism. In such cases a radionuclide with a comparatively short half-life added to a tracer containing a carrier substance is injected into the patient, e.g. ¹⁸F-FDG (Flourdeoxyglucose), which accumulates in specific organs and cell tissues and decays under emission of positrons. The accumulation preferably occurs in active cancer cells.

A positron released during the radioactive decay enters after a relatively short distance of typically one millimeter into interaction with an electron, with both particles being destroyed and two Gamma quanta with an energy of 511 keV in each case being emitted in a diametrically opposite direction. These annihilation quanta can be verified in space and time in a detector ring surrounding the object under examination or the patient, said ring comprising a plurality of gamma detectors arranged adjacently and able to be read out individually. A coincidence collimation in an evaluation unit connected downstream from the detectors allows the location of the electron position annihilation underlying the counter events to be determined on the imaginary line between the signal-emitting detector elements, known as the line of response. The gamma radiation is emitted isotropically, i.e. from a statistical standpoint all directions are equally probable. The spatial frequency distribution of the radioactive decay processes and thereby the distribution of the tracer in the body can be derived from a statistically significant plurality of counter events. Furthermore any given number of two-dimensional PET images can be calculated from this type of 3D volume data set.

PET involves a functional imaging which primarily attempts to record images of biochemical and physiological processes in the organism. In addition to allowing good analysis of the metabolism, it especially allows tumors and metastases to be found and the perfusion of the heart muscle to be assessed. However PET only has a relatively low local resolution (appr. 5 mm), which for basic reasons cannot be increased without imposing an additional radiation load on the patient. PET does not deliver good anatomical images, so that the spatial localization and assignment of the focus of the disease source detected produces difficulties.

Better anatomical images are available within the framework of computer tomography (CT), in which, from a plurality of x-ray images recorded from different directions, which are projection images in each case, cross-sectional images and 3D volume images are generated by computer-aided evaluation of the object under examination. The x-ray emitters and x-ray detectors required for this are arranged in the same configuration as for a PET detector ring, usually in a ring shape known as a gantry, in which case with new types of CT device only the x-ray emitters rotate in the gantry and the x-ray detectors distributed over 360° are arranged at fixed locations in the gantry in each case.

From The diagnostic standpoint it is frequently desirable to overlay the images obtained in a CT examination with the corresponding images from a PET examination. To enable a positionally-accurate simultaneous presentation or a (locationally) correct overlaying of the CT images with the PET images to be implemented an imaging specification between the co-ordinate systems underlying the images in each case must be found. This mapping can generally contain displacements, rotations and extensions, mostly even in combined form. This reconciliation process for the image data sets, which is also referred to as a registration, as a rule has only a restricted accuracy and despite extensive (computer-aided) automation frequently requires time-consuming user interaction. This is particularly true if the patient has to be moved into another room after the PET examination to carry out the subsequent CT examination, in which case under some circumstances his position or the attitude of his body changes and in which case his internal organs can move relative to one another.

To get around these types of difficulties of a purely-software-based registration, combined PET/CT systems have been developed in which a patient is moved on a patient table through PET and CT detector rings arranged immediately after one another. This is also referred to as a hardware-based registration of the image data or as a so-called “hard fusion”.

A significant disadvantage of this concept lies in the difficult access to the patient during examination caused by the two closed detector tubes being arranged adjacent one another. This type of arrangement can not only increase the unease of the patient, it is also as a rule not possible to undertake interventions during the examination procedure, e.g. minimally-invasive or surgical interventions. Instead the patient must be moved after the images have been recorded from the PET/CT scanner to a corresponding OP workstation. Here for example a suitable ablation catheter, which is introduced into a vessel in the body, can be used to undertake treatment of organic defects or focus of the disease previously identified with the aid of the PET/CT images. This treatment is frequently conducted under angiographic x-ray control, which necessitates a further imaging modality in the form of an angiographic x-ray diagnostic device. Such a device does provide silhouette-type images of the section of tissue affected, so that a certain level of control of the position and alignment of the oblation catheter within the vessel can be undertaken. However from the standpoint of the operator multiple “live images” of the region being treated with increased diagnostic information content are also desirable and precisely within the actual surgical or minimally-invasive treatment.

Although conventional angiography delivers high-resolution images of the vessel system, it is however not in a position to represent the surrounding soft tissue or any deposits on the wall of the vessel. If complications occur in the operation or during the catheter intervention it can thus be necessary to move the patient back into the PET/CT tomographs in order to produce a control image there. This means that under some circumstances valuable time for the initiation of the further therapy steps is lost, in which case the state of health of the patient may possibly drastically deteriorate.

A further development of x-ray angiography which delivers good 3D soft tissue images has recently been disclosed under the name “angiographic computer tomography” (ACT) or also “DynaCT” and is described for example in the older previously unpublished patent application 10 2004 057 308.5 or in the product brochure “Axiom Artis dFA—Axiom Artis dFA DynaCT—Universal, Floor-mounted C-arm angiographic system with Flat Detector” from Siemens AG, Medical Solutions from the year 2005, Order No. A91001-M1400-G938-2-7600. This further development is based on the one hand on an improved detector technology and on the other hand on improved image processing methods which refer back during the processing and visual conversion of the detector signals to special correction algorithms adapted to soft tissue display. Functional imaging is not possible with this type for apparatus for angiographic computer tomography, known by the abbreviated name of ACT modality.

### SUMMARY OF THE NVENTION

The object of the invention is thus to specify a medical imaging modality of the type mentioned at the start of which the images make a reliable detection and precise localization of tissue anomalies, especially of malign tissue with tumor incidences possible and which offers good access to the patient so that alongside the image recording operative and/or minimally-invasive interventions in the patient can be undertaken and checked.

The object is achieved in accordance with the invention by an ACT imaging device for angiographic computer tomography connected on the data side to an ACT image processing unit, being arranged adjacent to the PET and in which case the PET image processing unit and the ACT image processing unit is assigned to a common display unit for displaying the PET images and/or CT images generated in the relevant image processing unit.

The invention is based on the consideration that for a precise spatial localization of tumors, carcinomas, metastases etc. especially easily visible within the framework of the PET imaging, the PET images are to be supplemented by complementary morphological information of a further imaging method. By contrast with conventional CT images which are above all designed for a presentation of bones, organs and tissue structures, the additional image data should also include differentiated medical information about the blood vessels and their immediate environment, especially about adjacent soft tissue areas. This type of information is available with the aid of angiographic computer tomography (ACT).

To remove the difficulties usually associated with a software-based registration and fusion of different types of image right from the start and to avoid the necessity of transporting a patient between modalities at different locations, the PET unit and the ACT unit should be integrated into a combined PET/ACT modality, also referred to as a dual modality. Furthermore in such a modality the PET detector ring and the ACT imaging device comprising at least one x-ray emitter and one x-ray detector should be arranged so that a simultaneous recording of the PET images and the complementary ACT images, are at least one which subject to only a small time delay, is possible. To this end the ACT recording device is arranged adjacent to the PET detector ring, i.e. in relation to the longitudinal axis of the PET detector ring in front of or behind this ring. The patient, preferably fixed on a patient bed can then be moved in one pass and without any great interruption in time through the PET detector ring and subsequently through the ACT recording area and thus scanned. This simplifies the reconciliation of the PET images and the corresponding ACT images. The timing sequence of the images can also be reversed.

The detector signals acquired from the PET detector ring and the ACT x-ray detector are edited separately from one another in an image processing unit connected downstream on the data side of the relevant detector type and converted into PET images or ACT images. The image processing units can also be implemented as separate software modules of a common image processing computer. Finally the PET images and the ACT images can be displayed in a common display unit connected downstream from the PET image processing unit and the ACT image processing unit and this can preferably be done for the individual units alongside each other or below one another or if necessary also in overlaid or fused form. This means that the doctor undertaking the treatment can obtain all important medical information at a glance.

Preferably the PET image processing unit and the ACT image processing unit are connected on the data side to an image fusion unit so that a PET image can be overlaid or can be fused with a corresponding ACT image in (almost) real-time. The image fusing unit involved can be an independent image fusion computer or also a corresponding software module which can be run on a standard computer. The overlaid images are particularly informative for diagnostic purposes since the structural features of the organism under examination such as the skeleton or the organs for example, can be combined with functional information, e.g. about areas with diseased increased cell activity. The x-ray image data in this case to a certain extent forms a precise “map” in which the additional PET image data is embedded at the correct position.

The overlaying or fusion of the images can be undertaken in various ways: Comparatively simple to implement is a fusion of a 2D PET image with a corresponding 2D ACT image. Preferably the image fusion unit is however designed so that a fusion of the complete 3-dimensional volume data sets can be undertaken, in which case any 2-dimensional sectional images can subsequently be generated from the 3D fusion image and can be presented for display on the display unit.

Before the actual coalescence or overlaying of the PET images with the corresponding ACT images a reconciliation of the underlying co-ordinate systems in each case is expediently undertaken. To this end the image fusion unit advantageously features suitable means for a marker-based and or an image-based registration of the image data sets. With the marker-based registration the images to be overlaid are aligned with each other on the basis of common image elements, so-called markers, by translation and/or rotation and/or projection or scaling. The markers can be anatomical in origin or can also have been applied artificially. The identification and assignment of the markers is preferably undertaken automatically with the aid of suitable algorithms or also interactively in dialog with the user. In image-based registration the reconciliation of the images is undertaken on the basis of global morphological information, in which case suitable 2D or 3D correlation functions can be evaluated as a measure for the correspondence of the images. Since the PET unit and the ACT unit are integrated into a dual modality, the uniform co-ordinate system is specified by the patient support used for both units. This means that the registration of the image data sets and the image fusion can be undertaken with especially great precision and speed (hardware-based fusion).

To detect and take into consideration in the image fusion possible movements of the patient during the examination and especially in the short transition time during which the patient is moved from the PET detector unit to the ACT detector unit (or vice versa), the image fusion unit and is preferably connected on the data side to at least one movement sensor able to be fixed to the patient. The movement sensor can furthermore be connected to the PET image processing unit and/or the ACT image processing unit so that even before the fusion an appropriate correction or editing of the individual images to be fused can be undertaken. The registration and fusion of the individual images thus takes account of dynamic effects.

The movement sensor can operate according to an electrical, capacitive, magnetic, acoustic or optical principle and be embodied for wireless signal transmission, preferably in what is known as RFID (Radio frequency Identification) technology. For example the movement sensor in the form of an RFID microchip can be integrated into a plaster provided with an adhesive surface which is stuck onto the patient during examination and subsequently disposed of. Furthermore to record the movement of the patient, a movement sensor can be attached to the patient table or to the patient support. This movement sensor is also connected on the data side to an image fusion unit and/or to the relevant image processing unit (PET/ACT) so that the forwards movement can be taken into account in the image reconstruction and especially in the fused image reconstruction. In an additional or alternative embodiment a purely mathematical movement detection and correction based on a statistical evaluation of the image signals can also be provided in the image processor.

In addition to the movement sensors a number of physiological sensors connected on the data side to the relevant image processing unit (PET/ACT) and/or to the image fusion unit can advantageously be provided. These types of sensor can be embodied in particular to record organ movements such as the movement of the heart, the ribcage and the blood vessels. For example the breathing or the vessel pulsation can be measured in this way or an ECG can be recorded and be taken into consideration in the image reconstruction or the image fusion. The methods and algorithms which can be usefully employed for correction or elimination of such movement artifacts are well known to the person skilled in the art. The correction procedures implemented by software or hardware are also referred to as gating. To rectify the breathing artifacts for example a breast band can be used which determines via corresponding sensors the breathing amplitude and the breathing frequency. Alternatively the amplitude and the frequency can be computed from the envelope curve of the ECG signal and fed to a correction unit integrated into the image processing unit. In addition the pulsing of the vessels can be determined by evaluation of the ECG signal or the blood pressure curve.

In an expedient embodiment the ACT recording device of the PET/ACT modality comprises an x-ray source which can be moved on a track around a patient bed and an x-ray detector diametrically opposite the x-ray source which can be moved synchronously with the x-ray source on the circular track. It is not necessary for the devices to move through a complete 360° circle. In practice angular intervals of 180° to 220° are generally sufficient for image reconstruction. The x-ray detector is preferably a flat, especially rectangular or square semiconductor detector, with amorphous silicon advantageously being used as detector material.

Advantageously the x-ray source and the x-ray detector are arranged on a rotatable C-arm. The C-arm with the x-ray source and the x-ray detector rotates during image recording preferably by at least an angle of 180° around the patient, with projection images being recorded in rapid succession from various projection directions. The C-arm can be mounted on the floor or the ceiling. It can also be mounted on the arm of an industrial robot, especially of an industrial robot with five degrees of freedom, in which case a mounting which is adapted especially flexibly to different uses and to the relevant spatial circumstances is implemented. Provided it is not in use, the C-arm can also be hinged completely away from the examination area. The C-arm construction is especially advantageous since it occupies comparatively little space and guarantees good accessibility to the patient, so that If necessary treatment measures can be undertaken at the same time as the examination, e.g. surgical or minimally-invasive interventions. Alternatively the x-ray source and the associated x-ray detector can also be arranged on a ceiling or floor mounted tripod or another support structure, which can preferably be moved in three degrees of freedom around the patient.

Advantageously the medical imaging modality is constructed such that the patient can be moved into the system both from the PET side and also from the ACT side. To this end the patient expediently lies on a patient support equipped with a corresponding drive apparatus which allows automatic advance at preferably constant speed through the PET/ACT scanner.

Advantageously the PET subsystem and the ACT subsystem as well as jointly-used components of the modality are connected for the purposes of data interchange to a common system data bus. The common components, in addition to the image fusion unit and the display unit already mentioned, comprise a data memory, especially for storing the recorded image data, an input unit and a DICOM interface, via which an exchange of data with external modalities or with workstations connected to the Intranet of a hospital can be undertaken. Space and money can be saved by this multiple usage of a number of components. A common user interface which is adapted to a coordinated and tailored method of operation of the PET system and the ACT system also facilitates operation of the installation.

To avoid artifacts which may be produced by a possible overlaying of the individual detector signals (PET/ACT) it is further proposed to read out the image sensors offset in time and clocked. This is especially sensible if the patient is to be advanced without interruption from the PET scanner into the x-ray scanner.

Advantageously an ablation device is connected to the system data bus, with the data provided by the ablation device being able to be displayed on the display unit. The ablation device can especially be an aberration catheter with which diseased tissue is able to be removed within the framework of a minimally-invasive intervention. The ablation catheter features for this purpose for example a device for emitting laser rays (laser ablation) a feed system and an outlet system for a coolant (cryoablation) or means for emitting radio waves (radio wave ablation). As an alternative or in addition a feed system for a chemical, biological or pharmaceutical fluid can be provided for what is referred to as chemoembolization. Furthermore the catheter can be equipped with a number of physiological sensors and/or with an imaging sensor (intercorporal or intervascular imaging). The signals provided by these sensors can be edited in an evaluation unit and displayed on a monitor of the display unit. In particular a joint or overlaid display of images determined by intercorporal and extracorporal methods can be provided. The introduction and the handling of the ablation catheter can be undertaken under x-ray control, in which case the ACT unit of the combined PET/ACT modality is activated, providing CT-like images of the vessel system and of its environment. The PET subsystem is then expediently deactivated. This means that to perform the intervention the patient does not have to first be moved to an external angiography apparatus.

The benefits obtained with the invention lie particularly in the fact that the PET images and a CT images generated by the combined PET/ACT modality can be fused within a short time and with a high level of registration accuracy. In this case the advantages of a (functional) PET imaging oriented to presenting metabolistic processes are combined with the advantages of angiographic computer tomography (CT) which not only is able to display the vessel system of a human being at high resolution but is also able to map the surrounding soft tissue areas and thereby provides important additional medical information. A further advantage of the dual PET/ACT modality lies in the good accessibility to the patient for an accompanying tumor therapy for example. This advantage is based on the fact that the ACT imaging device, by contrast with conventional CT devices, does not need any enclosed gantries.

The application of the PET/ACT modality described within the framework of tumor ablation only represents one example of a medical application. Other therapies in which an anatomical and a functional imaging with a good access to the patient are necessary are also possible. For example the perfusion of the heart muscle can also be established with the PET and simultaneously with the ACT an anatomical assignment of the corresponding coronary vessels. Any necessary balloon dilation and stent implantation can be monitored with the aid of the ACT without a further imaging modality being needed for this purpose.

### BRIEF DESCRIPTION OF THE DRAWINGS

An exemplary embodiment of the invention is explained in more detail below with reference to a drawing. The Figures show:

FIG. 1 a schematic overview of a medical examination and treatment device (modality) with an integrated PET/ACT scanner,

FIG. 2 a PET image and a corresponding ACT image as well as a fusion image created by fusing the individual images, and

FIG. 3 a basic sketch which illustrates the clocked, time-offset reading out of detector signals.

### DETAILED DESCRIPTION OF THE INVENTION

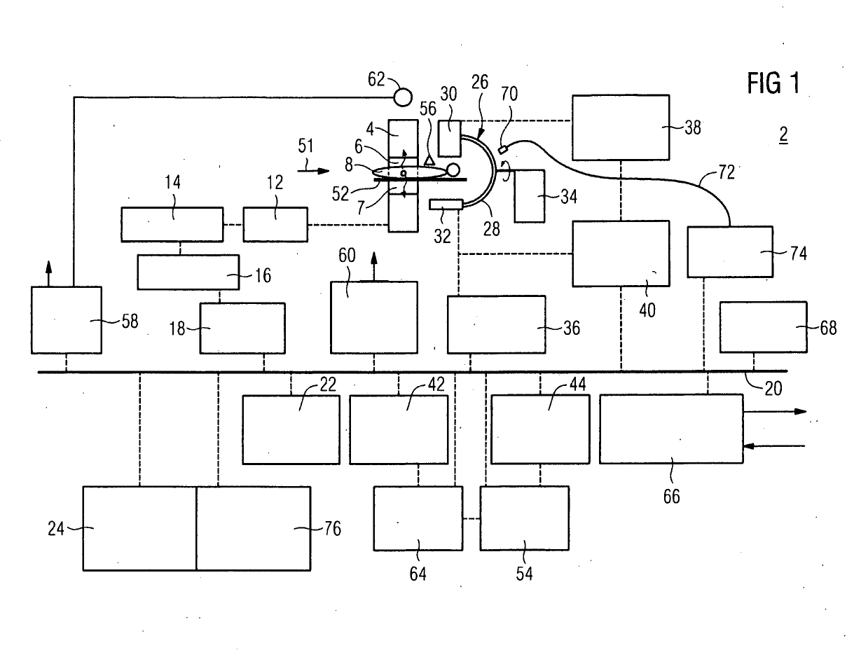

The medical examination and treatment device 2 shown in the schematic overview in FIG. 1, referred to in short below as a modality, comprises a PET unit based on the principle of Positron Emission Tomography (PET) The PET unit features a PET detector ring equipped with a plurality of scintillation or semiconductor detectors as well as with associated photo multipliers and pre-amplifiers to amplify the primary signals. The closed PET detector ring 4, shown in cross section here, is also referred to as a gantry. The detector elements of the PET detector ring 4 register—resolved in space and lime—in the cylindrical cavity 6 of the detector ring, energy-rich gamma quanta 7 emitted by a radiographic source. The radiographic source is in this case a human being, namely the patient 8 to be examined, into whom a slightly radioactive tracer is injected before the examination which accumulates in specific organs, especially in tumors, and which thus is distributed inhomogeneously in the body. The tracer most frequently used in PET is ¹⁸F-FDG (Fluordeoxyglucose). The applied quantity of the substance is extremely small and is in the sub-physiological range. There is thus no influence on the tissue change process to be investigated and no toxic reactions. The radionuclides necessary for producing the tracer are obtained in a reactor or cyclotron. Because of the short half-life of the radionuclides used in the PET method of for example two to then minutes, the reactor or the cyclotron is stationed in the vicinity of the medical examination and treatment device 2.

The decay of the radionuclide in the body of the patient 8 releases positrons which recombine within a very short space of time again with electrons. In each of these annihilation processes two gamma quanta 7 with an energy of 511 keV respectively are emitted in opposite directions (colinear). They then almost simultaneously—more precisely in a coincidence interval of approximately 10 ns—hit two diametrically opposed detector elements of the PET detector ring 4 and thus result in correlated timer events in the detector elements connected in coincidence. To obtain a volume of data relevant for significant statistical expressions, the timer events are integrated in time in an integrator 12 connected downstream from the detector ring 4 and subsequently analyzed in a pulse height analyzer 14 and a multi-channel analyzer 16.

Via a PET preprocessor 18 in which a correction of random and stray coincidences is undertaken, the PET detector signals edited in this way are fed to a system data bus for further distribution. In the PET image processing unit 22 the actual visual conversion of the PET signals in two PET images is undertaken in which the spatial distribution of the tracer in the organism of the patient is shown encoded by a color scale or by gray levels. In this case complete 3D volume data sets are preferably determined from which the 2D sectional images can be computed with any given sectional plane. The doctor performing the treatment can look at the PET images—if necessary after undertaking an artifact correction process to be described below—on a monitor of a display unit 24.

Because of the metabolic effectiveness of the tracer the PET imaging supplies valuable medical information about the metabolism processes taking place in the organism (functional imaging). However the anatomical assignment of the “hot spots” found in the PET images which for example are an indicator of tumors or metastases, is extremely difficult because of the comparatively low resolution of the PET methods and their lack of sensitivity for anatomical structures. For this reason a further imaging device is integrated into the imaging modality 2 which is able to provide image information which complements the PET images, which especially also covers the vessel system and the surrounding soft tissue. This involves a device for angiographic computer tomography (ACT), also known as “DynaCT”.

The ACT system in the exemplary embodiment comprises an ACT recording device arranged adjacent to the PET detector ring 4 with an x-ray source 30 arranged at one end of the C-arm 28 and with x-ray detector 32 arranged at the opposite end of the C-arm, which is embodied as a flat detector (matrix detector). Preferably amorphous silicon is used as a detector material here. Other materials can also be used, but as a rule of these usually make it necessary to use image amplifiers and more computing power. The C-arm 28 is supported to allow it to rotate on a floor-mounted stand 34 so that the x-ray emitter 30 and the associated x-ray detector 32 can be moved on an approximately circular track around the patient 8. To this end the corresponding rotation motors are integrated into the stand 34.

The patient located in the path of the rays of the x-ray emitter 30 causes, according to their x radiation transparency, a weakening of the x-rays which is detected by the x-ray detector 32. The detector signals read out from the x-ray detector 32 are processed in an x-ray preprocessor 36 and subsequently fed to the system data bus 20 for further distribution. The x-ray source 30 is supplied with the necessary operating voltage via a high-voltage generator 38. The high-voltage generator 38 is controlled by an x-ray system controller 40 which also co-ordinates the reading out of the x-ray detector 32. The x-ray system controller 40 also looks after the activation of the rotation motors for the C-arm 28 and synchronizes the rotation movement with the detection of the x-ray signals.

Two-dimensional sectional images which each represent a specific cross-section through the body of the patient 8 are recorded in an ACT image processing unit 42 from a plurality of projection images recorded during the rotational movement of the C-arm 28. Three-dimensional volume data sets are generated from a plurality of preferably “layered” or “stacked” sectional images in a 3D-reconstruction unit which is integrated into the ACT image processing unit 42 or can also be embodied as a separate component.

The ACT images can be displayed in the display unit 24 on their own or jointly with the corresponding PET images as 2D sectional images or as perspective 3D views. Particularly informative images are produced when the PET images are overlaid with the individual ACT images. To this end an image fusion unit 44 is connected to the system data bus 20, which reconciles the respective image data (registration) and builds on this to perform the actual fusion. In such cases complex 3D volume data sets are preferably fused. Alternatively there can also be provision for initially fusing a plurality of PET cross-sectional images with the corresponding ACT cross-sectional images in order to only subsequently construct from the 2D fusion images a 3D volume data set, i.e. a combined three-dimensional PET/ACT image. The fusion images can also be displayed on the common display unit 24.

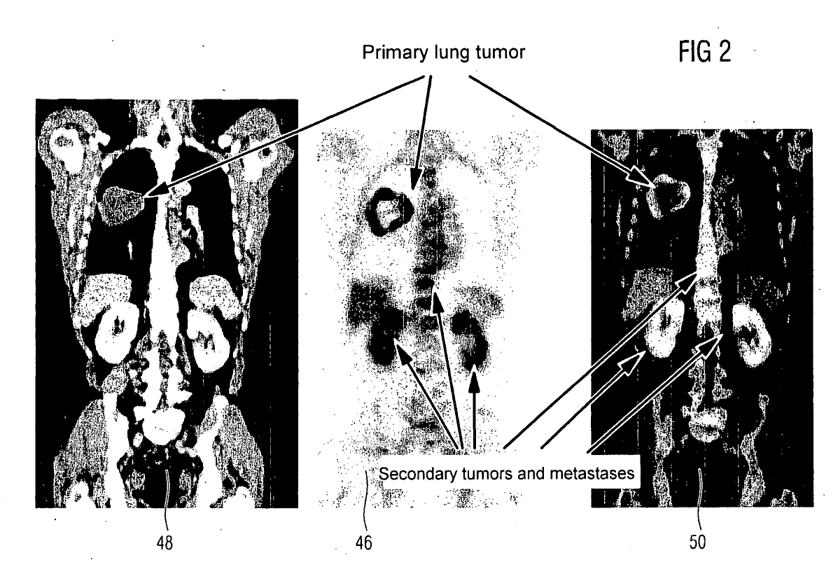

FIG. 2 shows a typical example of such an image fusion: A PET image 46 is shown in the center. The arrows indicate in the PET representation easily-visible primary tumors as well as secondary tumors and metastases. In the left-hand part of the figure the ACT image 48 corresponding to the PET image 46 with a high resolution and with differentiated reproduction of the soft tissue parts is shown, in which however the tumors are barely visible or only partly visible. The fusion image 50 shown on the right combines the advantages of the two individual images and allows the doctor performing the treatment to precisely anatomically assign the diseased areas.

Before the display of the individual images and/or the fusion images on the display monitor of the display unit, correction of image artifacts is expediently undertaken, especially of movement-induced image artifacts, e.g. caused by breathing, heart beat or the pulsing of vessels of the patient 8 or also by the forwards movement of the patient bed 52 indicated by the direction arrow 51. As can be seen in FIG. 1, an image correction unit 54 is connected to the system data bus 20 for this purpose. The artifact correction can already be undertaken at the level of the PET or ACT individual images, especially in the relevant 3D reconstruction. In particular correction algorithms are used in the editing of the ACT images which, in addition to correcting a movement-induced artifacts, are a good representation of soft tissue. These the types of algorithms are familiar to the person skilled in the art and can for example comprise a truncation correction, a stray radiation correction, an overradiation correction, a ring artifact correction, a correction of the beam hardening and the low frequency drop and/or a gain calibration.

Furthermore movement-induced artifacts, especially those which emanate from organ movements, can be taken into account and eliminated during image fusion. The correction unit 54 accesses in this case the sensor signals on the data input side of a number of position and movement sensors 56 and of physiological sensors not shown here in the drawing which are fed in via a movement and gating processor 58 and/or edited by a physiological signal processing unit 60 for further evaluation and fed into the system data bus 20. The electrophysiological sensors comprise sensors for pulse, respiration and blood pressure and also ECG electrodes. The position or movement sensors 56 are for example attached to the patient table 52 or directly to the patient 8. The sensors are at least partly embodied as RFID transponders which can be read out wirelessly via an assigned RFID reader or a signal receiver 62 and controlled if necessary. Before the start of the examination the movement sensor 56 must be calibrated in relation to the spatial co-ordinates of the examination apparatus. To do this a calibration unit 64 connected to the system data bus 20 is provided.

A DICOM interface 66 is connected to the system data bus 20 of the examination and treatment device 2 for communication with the outside world, said device being connected to a hospital information system (KIS) or to further imaging modalities or also to the Internet. DICOM (digital Imaging and Communications in Medicine) is an open standard for exchange of medical information, especially of image data and patient data. This type of data can be stored (buffered) before its flurther processing or transfer via the DICOM interface 66 in a data memory 68 connected to the system data bus 20.

Finally an ablation catheter 70 which can be introduced into the vessels or organs of the patient is connected via a data and power supply line 72 and an ablation catheter interface 74 to the data bus 20. The ablation catheter 70 allows treatment of the patient 8 to be undertaken at the same time or close to the time of the diagnostic imaging, e.g. a radio wave based tumor ablation. The ablation catheter 70 can be equipped with additional physiological or imaging sensors which are not shown in any greater detail here. The data provided in this way can also be converted visually and presented on the display unit 24, e.g. by insertion or overlaying with the images created in other ways.

A central input and output unit 76 which especially contains a keyboard, a computer mouse or an operator console, allows the user by means of the corresponding preferably menu-driven or dialog-based input operations, to control the entire medical examination and treatment device 2, including PET system, ACT x-ray system and ablation device. In this case all significant operations, examination protocols and frequently-used workflows are already predefined. After a workflow has been selected from one of the predefined selection lists, and if necessary after manual adjustment of individual parameters, the associated individual processes execute harmonized with one another or synchronized with each other automatically and largely without user interaction. The user can in this case, using the corresponding inputs at the input and output unit 76, influence how the images are displayed on the monitor of the display unit 24 and select expedient views or cross-sections.

A typical workflow for a purely diagnostic examination includes the following steps:
1. Injection of the tracer
2. Injection of x-ray contrast means.
3. Angiographic x-ray examination (ACT)
4. PET examination

Under some circumstances the injection of x-ray contrast means (step 2) can be dispensed with. The sequence of steps 3 and 4 can also be reversed. Thus a whole-body examination by means of PET can first be undertaken and then the ACT scan can be restricted to the organ area concerned which is highlighted in the PET images, which reduces the radiation load on the patient imposed by x-raying. During the examination the patient 8 is transported on the patient bed 52 fully automatically through the relevant examination areas (PET/ACT) of the combined modality.

A typical workflow for an examination with additional minimally-invasive therapy appears as follows:
1. Injection of the tracer
2. Injection of x-ray contrast means.
3. PET examination
4. Angiographic x-ray examination (ACT)
5. Introduction of the ablation catheter 70 under x-ray control (ACT)
6. Ablation of the tumor tissue
7. Checking the ablation with the aid of angiographic x-ray images.

It can also be sensible, before the PET/ACT examination, to create higher-resolution CT or MRI images in an external CT or MRI modality (MRI =Magnetic Resonance Imaging) and then to fuse these in a software-based process with the PET or ACT images or the combined PET/ACT images. Furthermore it is possible to conduct individual examinations with PET only or with ACT only. The subsystem not needed is then expediently deactivated.

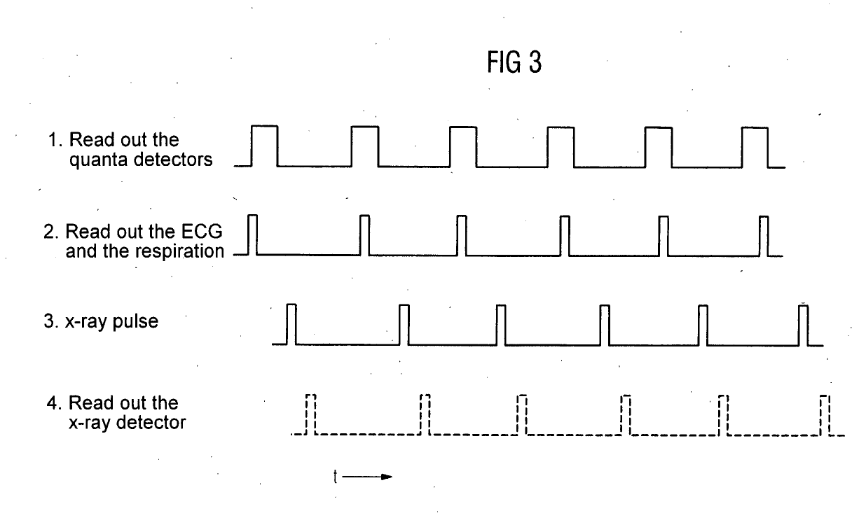

In order to exclude any undesired mutual influencing of the PET detector signals and the x-ray detector signals, the signal-emitting detectors 4, 32 are read out offset in time (clocked). This is illustrated schematically in FIG. 3. Taken in turn, the graphs, in which the abscissa represents the time t in each case, show from top to bottom:
1. The read-out intervals or time windows for the PET quanta detectors, shown by a rectangular signal shape above the level of the base line in each case,
2. The read-out intervals for the electrophysiological sensors, such as ECG or respiration sensors for example,
3. The time intervals in which the x-ray source 30 emits x-ray pulses, and
4. The read-out intervals for the x-ray detector 32.

The PET quanta detectors are essentially read out at the same time as the physiological sensors since this type of correlation is advantageous for artifact correction and gating. The x-ray pulses are generated offset in time in relation to these read-out processes. The x-ray detector is read out shortly after an x-ray pulse in each case, so that the read-out intervals for the x-ray detector 32 do not overlap with those for the PET detector ring 4. The frequency of the clocking can be set or configured.

1-13. (canceled)

14. A medical imaging modality used in a medical examination procedure of a patient, comprising:
a PET detector ring which records a raw positron emission tomography image data of the patient;
a PET image processing unit connected to the PET detector ring which generates a PET image from the recorded raw PET image data;
an ACT recording device adjacent to the PET detector ring which records a raw angiographic computer tomography image data of the patient;
an ACT image processing unit connected to the ACT recording device which generates an ACT image from the recorded raw ACT image data; and
a common display unit connected to the PET image processing unit and the ACT image processing unit which displays the PET image or the ACT image.

15. The medical imaging modality as claimed in claim 14, wherein the PET image processing unit and the ACT image processing unit are connected to an image fusion unit which generates a fused presentation of the PET image and the ACT image.

16. The medical imaging modality as claimed in claim 15, wherein the fused presentation of the PET image and the ACT image is generated from fusing a three-dimensional PET volume data set with a three-dimensional ACT volume data set.

17. The medical imaging modality as claimed in claim 15, wherein the fused presentation of the PET image and the ACT image is generated based on an image-based or a marker-based registration of the PET image with the ACT image.

18. The medical imaging modality as claimed in claim 15, wherein the image fusion unit is connected to a data input side of a sensor which detects a movement of the patient or an organ of the patient.

19. The medical imaging modality as claimed in claim 15, wherein the image fusion unit is connected to a data input side of a plurality of electrophysiological sensors.

20. The medical imaging modality as claimed in claim 15, wherein the common display unit displays the PET image or the ACT image individually or displays the fused presentation of the PET image and the ACT image.

21. The medical imaging modality as claimed in claim 14, wherein the ACT recording device comprises an x-ray source which moves on a circular track around a patient bed and an x-ray detector lying diametrically opposite to the x-ray source which moves synchronously with the x-ray source on the circular track.

22. The medical imaging modality as claimed in claim 21, wherein the x-ray detector is a flat semiconductor detector.

23. The medical imaging modality as claimed in claim 22, wherein the flat semiconductor detector comprises an amorphous silicon detector material.

24. The medical imaging modality as claimed in claim 21, wherein the x-ray source and the x-ray detector are supported on a C-arm for rotation.

25. The medical imaging modality as claimed in claim 21, wherein the patient bed is moved into an examination area of the medical image modality from both a recording area of the PET detector ring and a recording area of the ACT recording device by a drive element.

26. The medical imaging modality as claimed in claim 14,
wherein the medical image modality comprises a PET subsystem and an ACT subsystem,
wherein the PET subsystem and the ACT subsystem comprise a plurality of components respectively and a plurality of jointly-used components, and
wherein the PET subsystem, the ACT subsystem, and the jointly-used components are connected to a common system data bus.

27. The medical imaging modality as claimed in claim 26, wherein the jointly-used components comprise a DICOM interface, or an image and data memory, or an output unit.

28. The medical imaging modality as claimed in claim 26, wherein an ablation device is connected to the common system data bus and data recorded by the ablation device is displayed on the common display unit.

29. The medical imaging modality as claimed in claim 28, wherein the ablation device is an ablation catheter.

30. A method for performing a medical examination of a patient using a medical image modality, comprising:
recording a raw positron emission tomography image data of the patient by a PET detector ring;
generating a PET image from the recorded raw PET image data by a PET. image processing unit connected to the PET detector ring;
recording a raw angiographic computer tomography image data of the patient by an ACT recording device adjacent to the PET detector ring;
generating an ACT image from the recorded raw ACT image data by an ACT image processing unit connected to the ACT recording device;
connecting a common display unit to the PET image processing unit and the ACT image processing unit; and
displaying the PET image or the ACT image on the common display unit.

31. The method as claimed in claim 30, further comprising generating a fused presentation of the PET image and the ACT image by an image fusion unit connected to the PET image processing unit and the ACT image processing unit.

32. The method as claimed in claim 31, wherein the common display unit displays the PET image or the ACT image individually or displays the fused presentation of the PET image and the ACT image.
